(19) United States
(12) Patent Application Publication
(10) Pub. No.: US 2007/0233268 A1
(43) Pub. Date: Oct. 4, 2007

(54) Interpositional knee arthroplasty

(75) Inventors: Christel M. Wagner, Plymouth, IN (US); Daniel D. Auger, Fort Wayne, IN (US)

Correspondence Address: MAGINOT, MOORE & BECK, LLP;CHASE TOWER, 111 MONUMENT CIRCLE, SUITE 3250, INDIANAPOLIS, IN, 46204, US

(73) Assignee: DePuy Products, Inc., Warsaw, IN (US)

(21) Appl. No.: 11/395,617

(22) Filed: Mar. 31, 2006

(51) Int. Cl. A61F 2/38

(52) U.S. Cl. 623/20.14

(57) ABSTRACT

An interpositional knee arthroplasty is disclosed for attachment to a knee condyle. The interpositional knee arthroplasty comprises a spacer and at least one fastener extending from the spacer. The at least one fastener is designed and dimensioned to extend into the condyle without penetrating through the subcondylar plate. The spacer may be kidney shaped to facilitate attachment to the knee condyle. The fastener comprises at least one peg with at least one deformable fin attached to the peg. The deformable fin may be cup shaped. In operation, at least one hole is formed in a patient's condyle. The at least one hole is dimensioned to receive the at least one peg member and deformable fin, but the hole does not extend to the subcondylar bone. When the peg member is forced into the hole in the condyle, the deformable fin collapses and wedges the peg member in the hole.

### FIELD

This invention relates to the field of arthroplasty, and particularly to interpositional knee arthroplasty.

### BACKGROUND

Arthroplasty involves the surgical reconstruction or replacement of a malformed or degenerated joint. With interpositional arthroplasty, a knee implant is placed between inflamed joint surfaces to keep them apart. Such knee implants are often referred to as knee spacers.

In one common interpositional arthroplasty procedure, a knee spacer is placed between the tibia and the femur. In particular, the knee spacer is implanted between either the medial or lateral condyle of the femur and the meniscus of the tibia. The knee spacer provides a surface for articulation of the femur relative to the tibia.

Knee spacers are generally designed to conform to either the femur or the tibia in an attempt to prevent dislocation of the knee spacer within the joint. Various methods have been proposed for conforming the knee spacer within the joint. For example, some knee spacers are formed with a posterior lip that extends distally over the tibia. Other knee spacers are designed to conform to the femur in an attempt to retain the spacer within the joint. However, it has been noted that many of these knee spacers do not prevent in vivo movement of the knee spacer. In vivo movement of the knee spacer is one factor that may significantly contribute to the pain a patient experiences following an interpositional arthroplasty procedure.

Accordingly, it would be advantageous to provide a knee spacer for an interpositional knee arthroplasty that may be secured in such a manner to prevent in vivo movement of the knee spacer relative to the femur or tibia. It would be of further advantage if such knee spacer could be secured to the tibia in a manner that does not violate the subcondylar plate. It would also be advantageous if the knee spacer could be easily fixed to the femur or the tibia.

### SUMMARY

An interpositional knee arthroplasty is disclosed herein. The interpositional knee arthroplasty is configured for attachment to a lateral or medial condyle of a knee, each condyle including articular cartilage and subcondylar bone. The interpositional knee arthroplasty comprises a spacer and at least one fastener extending from the spacer. In one embodiment, the spacer comprises a kidney shaped plate. The fasteners are attached to one side of the spacer. Each fastener comprises a peg with at least one deformable fin attached to the peg. The deformable fins are cup shaped. In one embodiment, the spacer is also deformable such that a surface of the spacer may be contoured against the condyle.

In operation, a surgeon forms one or more holes in either the femoral or tibial condyle. In one embodiment, the holes do not perforate the subcondylar plate that forms the perimeter portion of the subcondylar bone. In this embodiment, the holes extend through the articular cartilage and into the subcondylar plate, but do not extend completely through the subcondylar plate. In an alternative embodiment, the holes formed by the surgeon extend completely through the subcondylar plate.

After the holes are formed in the condyle, the surgeon orients the spacer on the condyle with the fasteners directed toward the holes. The surgeon then presses against the spacer, forcing the fasteners into the holes. As the fasteners are forced into the holes, the deformable fins collapse. The collapsed fins act to wedge the pegs in the holes, thus securing the spacer to the condyle.

### BRIEF DESCRIPTION OF THE DRAWINGS

FIG. 1 shows a top view of an interpositional knee arthroplasty;

FIG. 2 shows a fastener for the interpositional knee arthroplasty of FIG. 1;

FIG. 3 shows a bottom view of the interpositional knee arthroplasty of FIG. 1;

FIG. 4 shows a side view of the interpositional knee arthroplasty of FIG. 1;

FIG. 5 shows a cross-sectional view of the interpositional knee arthroplasty along line V-V of FIG. 4;

FIG. 6 shows a perspective view of a femur and tibia with a first interpositional knee arthroplasty of FIG. 1 attached to the lateral plateau of the tibia and a second interpositional knee arthroplasty attached to the medial plateau of the tibia;

FIG. 7 shows a proximal view of the tibia of FIG. 6 showing the first interpositional knee arthroplasty positioned in the lateral plateau and the second interpositional knee arthroplasty positioned in the medial plateau; and

FIG. 8 shows a cross-sectional view of the tibia of FIG. 6 showing holes formed in the tibial condyles and the peg members extending into the subcondylar plate of the tibia.

### DESCRIPTION

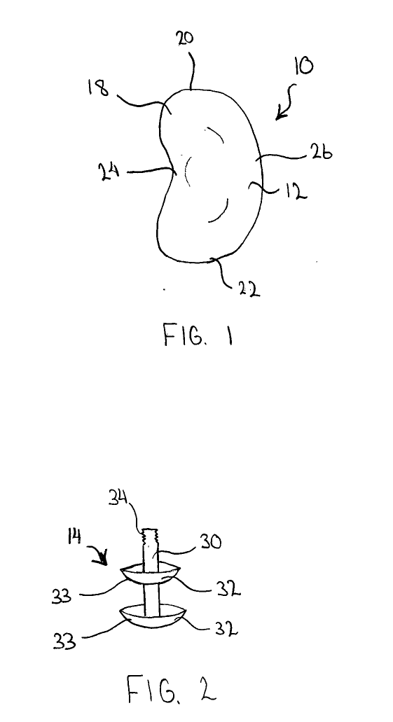

With general reference to FIGS. 1-5, an interpositional knee arthroplasty 10 comprises a spacer 12 and a plurality fasteners 14 extending from the spacer 12. As explained herein, the arthroplasty 10 is configured for attachment to either the lateral or medial plateau of the tibia.

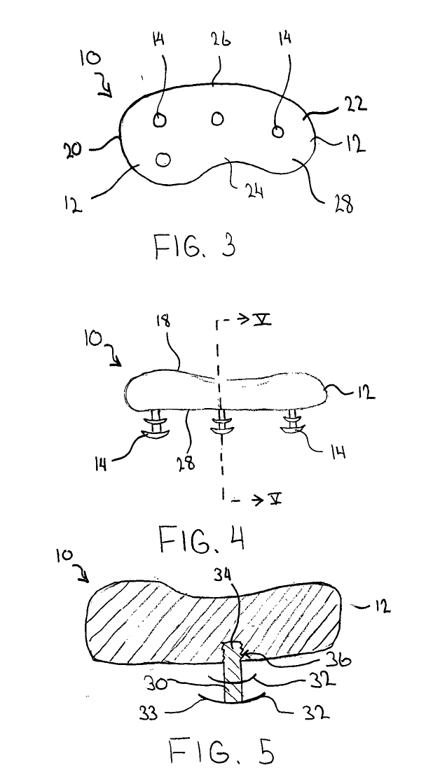

In the embodiment shown in FIGS. 1 and 3, the spacer 12 comprises a plate that is generally kidney shaped when viewed from the top and bottom. The spacer 12 is greater in length measured from its anterior side 20 to its posterior side 22 than it is in width measured from its medial side 24 to its lateral side 26. While the kidney shape of the spacer has advantages with respect to attachment of the spacer to the condyle, one of skill in the art will recognize that other shaped spacers may also be used.

The spacer 12 is comprised of a biocompatible material, either a polymer or metal such as ultra high molecular weight polyethylene (UHMWPE), polyurethane (PU), cobalt chrome (CoCr), or titanium (Ti). As shown in FIGS. 1 and 4, the top surface 18 of the spacer 12 is generally smooth with rolling contours. These rolling contours are designed to mimic the surface of a condyle facing the meniscus of the tibia.

As shown in FIGS. 3 and 4, the bottom surface 28 of the spacer 12 is generally flat with the fasteners 14 extending from the bottom surface 28. In one embodiment, the spacer 12 is somewhat flexible, allowing the bottom surface 28 to be bent in order to match the curved surface of the condyle when the spacer 12 is attached to the condyle. This distortion of the spacer 12 to match the curved surface of the condyle is more easily achieved because of the kidney shape of the spacer. In particular, the kidney shape of the spacer facilitates spacer distortion such that the bottom surface 28 more closely conforms to the curved surface of the condyle. In an alternative embodiment, the spacer 12 is more rigid, but the bottom surface 28 of the spacer is curved, allowing the bottom surface 28 of the spacer to conform to the curved surface of the condyle.

With reference to FIG. 4, the fasteners 14 may comprises peg members 15 that extend from the bottom surface 28 of the spacer 12. The plurality of peg members 15 each include a rigid center peg 30 and a plurality of deformable fins 32 attached to the center peg. In the disclosed embodiment, the deformable fins 32 comprise cup structures 33. Each cup structure 33 is secured at a central location to the center peg 30 and extends from such central location upward toward the bottom surface 28 of the spacer 12. A first cup structure 33 is positioned at the end of the center peg 30. A second cup structure is positioned at a midpoint of the center peg.

The cup structures 33 are secured to the center peg 30 by any of numerous methods. For example, the cup structures 33 may be secured to the center peg using adhesives or fasteners. In one embodiment, the center peg is comprised of a plurality of peg segments which are attached by one peg segment threadedly engaging an adjacent peg segment. In this arrangement, an extending screw portion of one peg segment is inserted through a center hole in a cup member and the adjacent peg sections are screwed together. This action clamps the center hole of the cup member between peg segments. Of course, numerous other methods may be used to secure the cup members 33 to the center peg, as will be recognized by those of skill in the art.

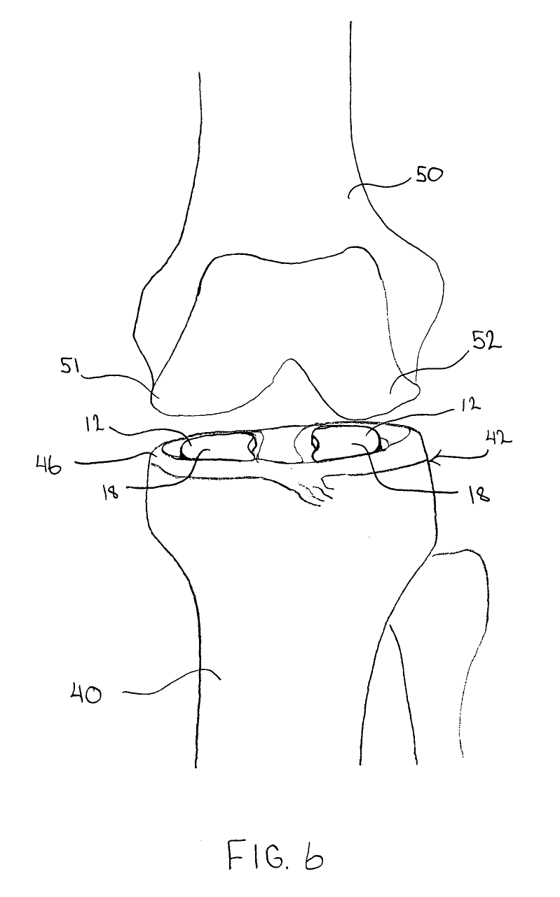

The center peg member is comprised of an appropriate rigid bio-compatible material, either a polymer or metal such as titanium or cobalt chromium. As shown in FIG. 5, each center peg 30 extends into the spacer 12 through the bottom surface 28, allowing the center peg to be fixed to the spacer. The center peg includes a threaded top portion 34. The threads on the top portion 34 of the center peg 30 engage threaded holes 36 in the bottom of the spacer to secure the center peg to the spacer. Adhesives may be used in the holes 36 of the spacer 12 to further secure the peg members 14 to the spacer 12. Of course, various other methods may be used to secure the center posts to the holes in the spacer, such as adhesives, friction fit, or snap fit arrangements, as well as other arrangements as will be recognized by those of skill in the art. Alternatively, the peg members may be integral with the spacer, such as an arrangement where the peg members are molded with the spacer as a one piece construction.

The deformable fins 32 or cup members 33 are designed in a manner and/or are comprised of an appropriate material that facilitates deformation of the fins. For example, the deformable fins 32 may be comprised of an appropriate deformable biocompatible material having relatively flexible characteristics, such as polyethylene or polyurethane. As explained in further detail below, the deformable cup members 33 are designed to collapse when forced into holes formed in the articular cartilage of the condyle. In alternative embodiments, the deformable fins 32 take different shapes other than that of cup members. For example, the deformable fins may comprise clover structures, spoked structures, circular structures that are not cupped, or numerous other designs. Furthermore, with certain designs, the deformable fins 32 may be comprised of a relatively rigid material instead of a flexible material. For example, in one embodiment, the deformable fins are comprised of a relatively rigid metal material arranged as a clover leaf cup structure. Such rigid metal fin structures will typically have a lesser thickness than fins comprised of more flexible material such as PE or PU.

With reference to FIG. 3, the plurality of peg members 15 are placed on the bottom surface 28 of the spacer 12 in an arrangement that promotes a secure attachment of the spacer 12 to the condyle. In the arrangement of FIG. 3, four peg members 15 are provided on the bottom surface 28 of the spacer 12. However, depending on numerous factors such as the size of the patient and particular shape of the spacer, different numbers of peg members may be used in different configurations.

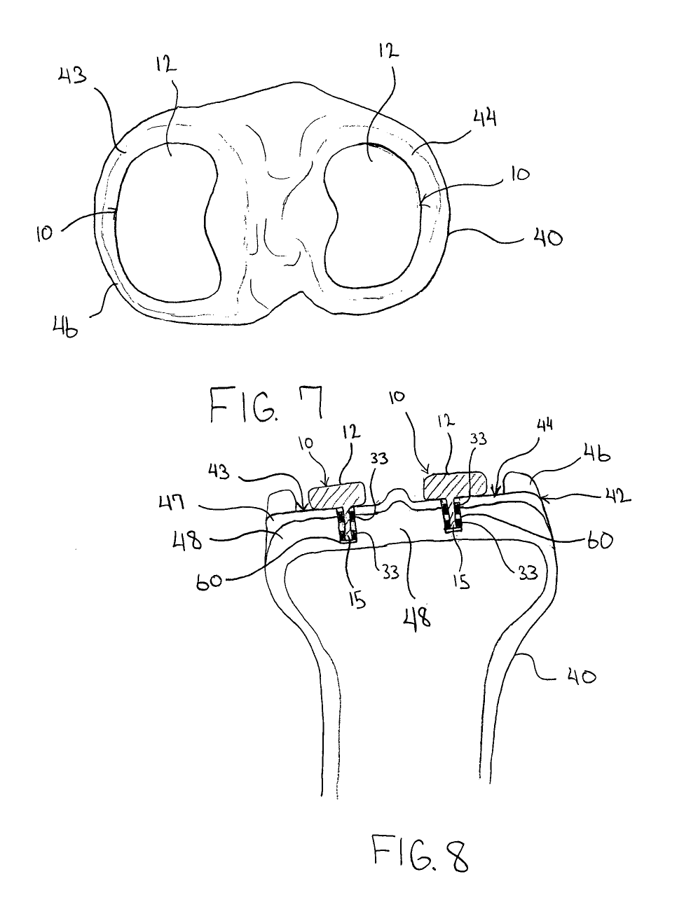

In order to secure the interpositional arthroplasty to either a femoral or tibial condyle, the surgeon first prepares the condyle by forming holes in the condyle. FIGS. 6-8 show an exemplary embodiment where an interpositional arthroplasty 10 is secured to each tibial condyle 43 and 44. FIG. 8 particularly shows a cross-sectional view of the tibia 40 with holes 60 formed in the tibial condyles 43 and 44.

Before the holes 60 are formed in the condyles 43 and 44, the surgeon first clears the interior portion of the meniscus 46. With the interior of the meniscus 46 cleared, the medial tibial condyle 43 and lateral tibial condyle 44 are open to receive an arthroplasty 10. In one embodiment, the surgeon may also smooth the exterior articular cartilage 47 to prepare a surface for the spacer 12.

Once the interior portion of the meniscus 46 is cleared, holes 60 are formed in the condyles 43 and 44 using a drill and guide arrangement. Such arrangements are common in prosthetic procedures. In this case, the drill and guide arrangement is configured to create a hole 60 in the condyle that extends to the subcondylar plate 48. In one embodiment, the depth of the hole extends through the articular cartilage 47, but does not extend completely through the subcondylar plate 48. The actual depth of such a hole will depend upon the patient, but the typical depth of such a hole in the condyle that does not extend through the subcondylar plate 48 is less than eight mm. In an alternative embodiment, the hole created by the surgeon in the condyle extends completely through the subcondylar plate 48, thus perforating the subcondylar plate. In any event, the diameter of the hole that is created in the condyle is slightly larger than the diameter of the center peg of a peg member, but smaller than the diameter of deformable a cup member 33. This allows the peg member to be inserted into the hole while causing the cup members 33 to collapse.

After the holes are formed in the condyles, the surgeon aligns the peg members 15 with the holes in the condyle and manually presses against the spacer 12 to force the peg members 15 into the holes. FIG. 8 shows the peg members 15 inserted in the holes 60. When the deformable cup members 33 are forced into the holes in the condyle along with a peg member 15, the deformable cup members 33 collapse since the diameter of the hole 60 is less than the diameter of the cup members 33. The deformed cup members 33 are collapsed into excessive space in the hole and wedge the center peg in the hole in a friction fit arrangement. The deformed cup members are represented in FIG. 8 by the rectangular portions positioned between the holes 60 and the peg members 15. The wedging action of the deformed cup members effectively secures the arthroplasty to the associated condyle.

Following implantation, the spacer 12 of each arthroplasty 10 is secured to a condyle 43 or 44 and covers a substantial portion of the exterior surface of the condyle. In particular, each spacer 12 is configured to cover the articular cartilage 47 surface portion of the condyle that would normally contact the meniscus 46 of the tibia 44. Thus, as shown in FIG. 6, the top surface 18 of each spacer 12 is exposed to the femur 50, with each top surface 18 directly opposed to a femoral condyle 51, 52. With the spacers 12 in place, inflamed joint surfaces on the femur and tibia are separated, thus relieving pain encountered by the patient. Furthermore, with the arthroplasty secured to the condyle, in-vivo movement of the arthroplasty is restricted, and less pain is encountered by the patient following the procedure. Also, because the arthroplasty may be secured without perforating the subcondylar plate, the patient may experience less pain following surgery along with a faster recovery time.

While FIGS. 6-8 shown an interpositional knee arthroplasty 10 attached to both the medial tibial condyle 43 and the lateral tibial condyle 44, some surgeries may only call for the use of a single arthroplasty secured to a single condyle, depending on the needs of the patient. Furthermore, as mentioned above, the interpositional knee arthroplasty may be secured to either the tibial condyles or the femoral condyles.

Although the present invention has been described and shown with respect to certain preferred embodiments, it will be appreciated by those of skill in the art that other implementations and adaptations are possible. For example, although the pegs have been shown in the figures extending in a perpendicular fashion from the spacer, the pegs may also be positioned at an acute angle, such that the pegs enter the condyle at an associated angle. As another example, different shaped or different numbers of fins may be attached to each peg. Moreover, in addition to alternative embodiments, there are advantages to individual advancements described herein that may be obtained without incorporating other aspects described above. Therefore, the spirit and scope of the appended claims should not be limited to the description of the preferred embodiments contained herein.

What is claimed is:

1. An interpositional knee arthroplasty configured for attachment to a condyle which includes articular cartilage and a subcondylar plate, the interpositional knee arthroplasty comprising:
a) a spacer; and
b) at least one fastener extending from the spacer, the at least one fastener designed and dimensioned to extend through the articular cartilage and into the subcondylar plate.

2. The interpositional knee arthroplasty of claim 1 wherein the at least one fastener is designed and dimensioned to extend into the subcondylar plate without perforating the subcondylar plate.

3. The interpositional knee arthroplasty of claim 1 wherein the at least one fastener is designed and dimensioned to extend through the subcondylar plate.

4. The interpositional knee arthroplasty of claim 1 wherein the spacer comprises a kidney shaped plate.

5. The interpositional knee arthroplasty of claim 1 wherein the fastener comprises at least one deformable fin.

6. The interpositional knee arthroplasty of claim 5 wherein the fastener further comprises a peg, and the at least one deformable fin is attached to the peg.

7. The interpositional knee arthroplasty of claim 5 wherein the deformable fin comprises a cup member.

8. The interpositional knee arthroplasty of claim 1 wherein the spacer is configured for positioning on a lateral condyle.

9. The interpositional knee arthroplasty of claim 1 wherein the spacer is configured for positioning on a medial condyle.

10. The interpositional knee arthroplasty of claim 1 wherein the spacer is deformable such that a surface of the spacer may be contoured against the condyle.

11. A method of providing an interpositional knee arthroplasty comprising:
a) providing a spacer having at least one fastener extending from the spacer;
b) forming at least one hole in a condyle, the at least one hole extending through articular cartilage and into a subcondylar plate;
c) attaching the spacer to the condyle by inserting at least one fastener into the at least one hole.

12. The method of claim 11 wherein the at least one hole does not perforate the subcondylar plate.

13. The method of claim 11 wherein the at least one hole perforates the subcondylar plate.

14. The method of claim 11 wherein the fastener comprises at least one fin, and wherein the step of attaching the spacer to the condyle includes deforming the at least one fin as the at least one fastener is inserted into the at least one hole.

15. The method of claim 11 wherein the step of attaching includes pressing on the spacer to force the at least one fastener into the at least one hole.

16. The method of claim 11 wherein the spacer comprises a kidney shaped plate.

17. The method of claim 11 wherein the fastener comprises at least one cup member attached to a peg.

18. An interpositional knee arthroplasty configured for attachment to a knee condyle, the interpositional knee arthroplasty comprising:
a) an implant plate configured for attachment to the knee condyle;
b) at least one peg extending from the implant plate; and
c) at least one deformable fin attached to the at least one peg.

19. The interpositional knee arthroplasty of claim 18 wherein the deformable fin comprises a cup member.

20. The interpositional knee arthroplasty of claim 18 wherein the at least one peg threadedly engages the implant plate.

21. The interpositional knee arthroplasty of claim 18 wherein the implant plate is kidney shaped.

22. The interpositional knee arthroplasty of claim 18 is designed and dimensioned to extend into the condyle such that it does not extend to the subcondylar bone.

23. The interpositional knee arthroplasty of claim 18 wherein the implant plate is configured for attachment to a lateral condyle.

24. The interpositional knee arthroplasty of claim 18 wherein the implant plate is configured for attachment to a medial condyle.
